(19) United States
(12) Patent Application Publication
(10) Pub. No.: US 2013/0245050 A1
(43) Pub. Date: Sep. 19, 2013

(54) Use of N-Phenylethylpyraozole Carboxamide Derivatives or Salts Thereof for Extending Shelf Life of Fruits and Vegetables

(71) Applicants: Heiko Rieck, Burscheid (DE); Dominique Steiger, Dusseldorf (DE); Peter Howard Davies, Ayala Alabang Village (PH); Sylvain Tafforeau, Lyon (FR); Lorianne Fought, Kerman, CA (US); George Hauley Musson, IV, Raleigh, NC (US); Luk De Maeyer, Linter (BE); Gilbert Labourdette, Paray Le Monial (FR); Helen Lachaise, Lyon (FR); Sebastian Hoffmann, Neuss (DE)

(72) Inventors: Heiko Rieck, Burscheid (DE); Dominique Steiger, Dusseldorf (DE); Peter Howard Davies, Ayala Alabang Village (PH); Sylvain Tafforeau, Lyon (FR); Lorianne Fought, Kerman, CA (US); George Hauley Musson, IV, Raleigh, NC (US); Luk De Maeyer, Linter (BE); Gilbert Labourdette, Paray Le Monial (FR); Helen Lachaise, Lyon (FR); Sebastian Hoffmann, Neuss (DE)

(73) Assignee: BAYER INTELLECTUAL PROPERTY GMBH, MONHEIM (DE)

(21) Appl. No.: 13/817,052

(22) PCT Filed: Aug. 15, 2011
(86) PCT No.: PCT/EP2011/064006

§ 371 Date: May 28, 2013

(30) Foreign Application Priority Data
Aug. 17, 2010 (EP) 10173071.1
Apr. 7, 2011 (EP) 11161526.6

(51) Int. Cl. C07D 231/14; A01N 43/54; A01N 37/50; A01N 43/56

(52) U.S. Cl. CPC C07D 231/14; A01N 43/56; A01N 43/54; A01N 37/50
USPC 514/269; 548/374.1; 514/406

(57) ABSTRACT

The present invention relates to the use of N-phenylethylpyrazole carboxamide derivatives or salts thereof for extended shelf life and storage stability of fruits and vegetables, to a method for extending shelf life or fruits and vegetables by applying N-phenylethylpyrazole carboxamide derivatives or salts thereof to the crops prior to the harvest of the fruits or vegetables.

The present invention relates to the use of N-phenylethylpyrazole carboxamide derivatives, in particular 3-difluoromethyl-1-methyl-1H-pyrazole-4-carboxylic acid [2-(2,4-dichlorophenyl)-2-methoxy-1-methyl-ethyl]-amide for extending shelf life and storage stability of fruits and vegetables and to a method for extending shelf life of fruits and vegetables by applying N-phenylethylpyrazole carboxamide derivatives or its salts to the crops prior to the harvest of the fruits or vegetables.

Fresh fruits and vegetables are extremely perishable commodities. Heretofore many techniques have been employed to protect such food products from oxidative degradation, mold attack and moisture penetration and to preserve the freshness, texture and color of the fresh produce.

One of the earliest means of lengthening the shelf life of fruits and vegetables was refrigeration. However, most fresh produce when stored under reduced temperatures for prolonged periods shows adverse effects on the taste, odor or quality of the product from microbial and mold growth above 1.7° C. In addition, storage temperatures below 1.7° C. often show chill injury to the tissue of the produce. Hence, in many instances refrigeration alone is not effective in achieving the desired shelf life for a particular fruit or vegetable.

Coating fresh fruits and/or vegetables is another of these techniques which has been employed with varying degrees of success. Not only must the coating be effective in prolonging the useful shelf life of the fresh product, but the appearance of the commodity must not be altered from that of its natural state. At a minimum, this natural appearance must not only remain unchanged but should ideally be enhanced especially when the fruit or vegetable will be displayed for sale. The selection of a coating material is further complicated where the fruit or vegetable is to be consumed in its natural state and it is considered essential that there be no need to remove the coating. In that event, the coating material must not only be edible, it must not affect or alter the natural organoleptic characteristics of the fresh fruit or vegetable.

Food preservation has for many years employed such mutually exclusive processes as dehydration and freezing. Both of these operations often include a heat treatment, known as blanching, which is conducted prior to the dehydration or freezing step. Blanching is said to reduce enzyme or bacteria level and to prevent or minimize undesirable changes during storage in the dry or frozen state, such as changes in color, odor or texture or loss of vitamins. Blanching may be conducted with steam (e.g., U.S. Pat. No. 2,373,521 of Wigelsworth), hot water (U.S. Pat. No. 2,515,025 of Vahl et al.), hot oxygen-free gas (U.S. Pat. No. 3,801,715 of Smith et al.) or hot air (U.S. Pat. No. 3,973,047 of Linaberry et al.)

Therefore, there is a strong need for methods for improving shelf life of fruits and vegetables which avoid the drawbacks resulting from the prior art methods.

The problem outlined above has been solved by the use of N-phenylethylpyrazole carboxamide derivatives or its salts for extending shelf life of fruits and vegetables.

It has surprisingly been found that the application of N-phenylethylpyrazole carboxamide derivatives, in particular 3-difluoromethyl-1-methyl-1H-pyrazole-4-carboxylic acid [2-(2,4-dichlorophenyl)-2-methoxy-1-methyl-ethyl]-amide during the growing and maturation periods avoids development of bacterial or fungal diseases during the storage period. This effect was especially notable with the reduction of one of the major fungus causing post harvest problems, i.e. Rhizopus spp. Consequently, shelf life and storage stability of the treated fruits and vegetables is significantly extended.

### DETAILED DESCRIPTION OF THE INVENTION

In conjunction with the present invention N-phenylethylpyrazole carboxamide derivatives or salts thereof, are represented by the general formula (I)

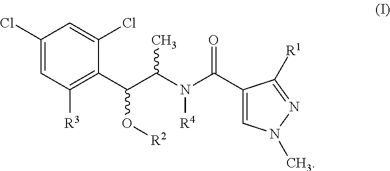

In this formula,

R³ represents a halo(C₁-C₆)alkyl group, preferably CHF₂ or CF₃;

R² represents a (C₁-C₆)alkyl group, preferably methyl or ethyl;

R³ represents a hydrogen or halogen atom, preferably hydrogen or chloro;

R⁴ represents hydrogen or a (C₃-C₆)cycloalkyl group, preferably hydrogen or cyclopropyl.

In a most preferred embodiment of the invention the N-phenylethylpyrazole carboxamide derivative to be used is 3-difluoromethyl-1-methyl-1H-pyrazole-4-carboxylic acid [2-(2,4-dichlorophenyl)-2-methoxy-1-methyl-ethyl]-amide. 3-Difluoromethyl-1-methyl-1H-pyrazole-4-carboxylic acid [2-(2,4-dichlorophenyl)-2-methoxy-1-methyl-ethyl]-amide usually is a mixture of 4 different stereo isomers. Processes suitable for its preparation from commercially available starting materials are described in WO 2008/148570. The different stereo isomers (+)-3-difluoromethyl-1-methyl-1H-pyrazole-4-carboxylic acid [(1R,2 S)-2-(2,4-dichlorophenyl)-2-methoxy-1-methyl-ethyl]-amide, (−)-3-difluoromethyl-1-methyl-1H-pyrazole-4-carboxylic acid [(1 S,2R)-2-(2,4-dichlorophenyl)-2-methoxy-1-methyl-ethyl]-a mide; (−)-3-difluoromethyl-1-methyl-1H-pyrazole-4-carboxylic acid[(1R,2R)-2-(2,4-dichlorophenyl)-2-methoxy-1-methyl-ethyl]-amide and (+)-3-difluoromethyl-1-methyl-1H-pyrazole-4-carboxylic acid [(1S,2S)-2-(2,4-dichlorophenyl)-2-methoxy-1-methyl-ethyl]-amide can be separated, for example by HPLC, using a chiral stationary phase column, as described in WO 2010/000612. All those 4 stereoisomers showing excellent activity according to the invention.

According to the invention also the agronomically acceptable salts/isomers/enatiomers and N-oxides of the compounds according to formula I, in particular of 3-difluoromethyl-1-methyl-1H-pyrazole-4-carboxylic acid [2-(2,4-dichlorophenyl)-2-methoxy-1-methyl-ethyl]-amide, can be used for extending shelf life of fruits and vegetables.

In conjunction with the present invention shelf life (or storability) denotes the period of time during which the perishable fruit or vegetable can be stored refrigerated or un-refrigerated, and remain edible and free from noticeable or harmful degradation or contamination by microorganisms or fungi.

Extension of shelf life denotes an elongation of shelf life of at least several days, preferably of at least one week, most preferably of at least one month.

The use/method according to the present invention can be applied to any kind of fruits and vegetables.

Examples for fruits are banana, blackcurrant, redcurrant, gooseberry, tomato, eggplant, guava, lucuma, chili pepper, pomegranate, kiwifruit, grape, table grapes, pumpkin, gourd, cucumber, melon, orange, lemon, lime, grapefruit, banana, cranberry, blueberry, blackberry, raspberry, boysenberry, hedge apple, pineapple, fig, mulberry, apple, apricot, peach, cherry, green bean, sunflower seed, strawberry and plum.

Examples for vegetables are flower buds, such as: broccoli, cauliflower, globe artichokes; seeds, such as sweetcorn also known as maize; leaves, such as kale, collard greens, spinach, beet greens, turnip greens, endive; leaf sheaths, such as leeks; buds, such as Brussels sprouts; stems of leaves, such as celery, rhubarb; stem of a plant when it is still a young shoot, such as asparagus, ginger; underground stem of a plant, also known as a tuber, such as potatoes, Jerusalem artichokes, sweet potato, yam; whole immature plants, such as bean sprouts; Roots, such as carrots, parsnips, beets, radishes, turnips; bulbs, such as onions, garlic, shallots.

In a preferred embodiment strawberries or table grapes are treated with N-phenylethylpyrazole carboxamide derivatives or salts thereof, most preferably with 3-difluoromethyl-1-methyl-1H-pyrazole-4-carboxylic acid [2-(2,4-dichlorophenyl)-2-methoxy-1-methyl-ethyl]-amide or its derivatives or its salts in order to improve the shelf live of the harvested fruits.

The N-phenylethylpyrazole carboxamide derivative or its salts, preferably 3-difluoromethyl-1-methyl-1H-pyrazole-4-carboxylic acid [2-(2,4-dichlorophenyl)-2-methoxy-1-methyl-ethyl]-amide or its derivatives or its salts, can be employed for extending shelf life of fruits and vegetables within a certain period of time after the treatment of the crops bearing the fruits or vegetables or after treating the fruits or vegetables itself. Generally, the

N-phenylethylpyrazole carboxamide derivatives or its salts is applied to the crop or to its fruits or vegetables prior to the harvest, more preferably prior to the maturation of fruits and vegetables, most preferably during the plant and fruit growth prior to contamination event.

The period of time within which protection is effected generally extends from 1 hour to 6 months, preferably from 1 week to 1 month after the treatment of the crops or its fruits or vegetables with the active compounds.

When employing the N-phenylethylpyrazole carboxamide derivatives or its salts, preferably 3-difluoromethyl-1-methyl-1H-pyrazole-4-carboxylic acid [2-(2,4-dichlorophenyl)-2-methoxy-1-methyl-ethyl]-amide or its derivatives or salts, according to the present invention for extending the self life of the harvested fruits or vegetables, the application rates can be varied within a broad range, depending on the type of application. For foliar applications the application rates of active compound are generally ranging from 1 to 250 g/ha, more preferably from 25 to 200 g/ha, most preferably from 30 to 150 g/ha based upon the pure a.s. (active substance).

According to the present invention the N-phenylethylpyrazole carboxamide derivatives or its salts, preferably 3-difluoromethyl-1-methyl-1H-pyrazole-4-carboxylic acid [2-(2,4-dichlorophenyl)-2-methoxy-1-methyl-ethyl]-amide or its derivatives or salts, can be applied to all parts of the plants such as shoot, leaf, flower and root, leaves, needles, stalks, stems, flowers, vegetative buds and flower buds fruiting bodies and fruits.

Plants are understood as meaning, in the present context, all plants and plant populations, such as desired and undesired wild plants or crop plants (including naturally occurring crop plants). Crop plants or crops may be plants which can be obtained by conventional breeding and optimization methods or else by biotechnological and genetic engineering methods or by combinations of these methods, including the transgenic plants and including the plant varieties capable or not capable of being protected by plant breeders' rights.

According to the invention the treatment of the plants with the N-phenylethylpyrazole carboxamide derivatives or its salts, preferably 3-difluoromethyl-1-methyl-1H-pyrazole-4-carboxylic acid [2-(2,4-dichlorophenyl)-2-methoxy-1-methyl-ethyl]-amide or its derivatives or salts, is carried out directly by the customary treatment methods, for example by immersion, spraying, vaporizing, fogging, injecting, dripping, drenching, broadcasting or painting. In a preferred embodiment of the invention 3-difluoromethyl-1-methyl-1H-pyrazole-4-carboxylic acid [2-(2,4-dichlorophenyl)-2-methoxy-1-methyl-ethyl]-amide is applied by injecting, dripping, drenching or spraying.

The N-phenylethylpyrazole carboxamide derivatives or its salts, preferably 3-difluoromethyl-1-methyl-1H-pyrazole-4-carboxylic acid [2-(2,4-dichlorophenyl)-2-methoxy-1-methyl-ethyl]-amide or its derivatives or salts can be converted to the customary formulations, such as solutions, emulsions, suspensions, powders, foams, pastes, granules, aerosols, very fine capsules in polymeric substances and in coating compositions for seed, and also ULV cold- and warm-fogging formulations.

These formulations are produced in a known manner, for example by mixing the active compounds with extenders, that is liquid solvents, pressurized liquefied gases and/or solid carriers, optionally with the use of surface-active agents, that is emulsifiers and/or dispersants and/or foam formers. If the extender used is water, it is also possible to employ for example organic solvents as cosolvents. Suitable liquid solvents are essentially: aromatics, such as xylene, toluene or alkylnaphthalenes, chlorinated aromatics or chlorinated aliphatic hydrocarbons, such as chlorobenzenes, chloroethylenes or methylene chloride, aliphatic hydrocarbons, such as cyclohexane or paraffins, for example mineral oil fractions, alcohols, such as butanol or glycol as well as their ethers and esters, ketones, such as acetone, methyl ethyl ketone, methyl isobutyl ketone or cyclohexanone, strongly polar solvents, such as dimethylformamide and dimethyl sulphoxide, and also water. Liquefied gaseous extenders or carriers are those liquids which are gaseous at ambient temperature and at atmospheric pressure, for example aerosol propellants such as halogenated hydrocarbons and also butane, propane, nitrogen and carbon dioxide. As solid carriers there are suitable: for example ground natural minerals, such as kaolins, clays, talc, chalk, quartz, attapulgite, montmorillonite or diatomaceous earth, and ground synthetic minerals, such as finely divided silica, alumina and silicates. As solid carriers for granules there are suitable: for example crushed and fractionated natural rocks such as calcite, pumice, marble, sepiolite and dolomite, and also synthetic granules of inorganic and organic meals, and granules of organic material such as sawdust, coconut shells, maize cobs and tobacco stalks. As emulsifiers and/or foam formers there are suitable: for example non-ionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and protein hydrolysates. As dispersants, for example, lignosulphite waste liquors and methylcellulose are suitable.

Tackifiers such as carboxymethylcellulose and natural and synthetic polymers in the form of powders, granules or latices, such as gum arabic, polyvinyl alcohol and polyvinyl acetate, as well as natural phospholipids, such as cephalins and lecithins, and synthetic phospholipids, can be used in the formulations. Other possible additives are mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations in general contain between 0.1 and 95 percent by weight of active compounds, preferably between 0.5 and 90 percent by weight, based upon the total formulation.

According to the present invention, the N-phenylethylpyrazole carboxamide derivatives or its salts, preferably 3-difluoromethyl-1-methyl-1H-pyrazole-4-carboxylic acid [2-(2,4-dichlorophenyl)-2-methoxy-1-methyl-ethyl]-amide or its derivatives or salts as such or their formulations, can also be used as a mixture with known fungicides, bactericides, acaricides, nematicides, or insecticides, for example, to broaden the activity spectrum or prevent the development of resistance. In many instances, synergistic effects are obtained, i.e. the activity of the mixture exceeds the activity of the individual components.

A further embodiment of the invention relates to the use of a composition comprising of N-phenylethylpyrazole carboxamide derivatives, preferably of 3-difluoromethyl-1-methyl-1H-pyrazole-4-carboxylic acid [2-(2,4-dichlorophenyl)-2-methoxy-1-methyl-ethyl]-amide and a second fungicide extending shelf life of fruits and vegetables.

Suitable fungicides which can be used in combination with the N-phenylethylpyrazole carboxamide derivatives or its salts, preferably 3-difluoromethyl-1-methyl-1H-pyrazole-4-carboxylic acid [2-(2,4-dichlorophenyl)-2-methoxy-1-methyl-ethyl]-amide or its derivatives or salts are selected from the group consisting of
(1) Inhibitors of the nucleic acid synthesis, for example benalaxyl, benalaxyl-M, bupirimate, clozylacon, dimethirimol, ethirimol, furalaxyl, hymexazol, metalaxyl, metalaxyl-M, ofurace, oxadixyl and oxolinic acid.
(2) Inhibitors of the mitosis and cell division, for example benomyl, carbendazim, chlorfenazole, diethofencarb, ethaboxam, fuberidazole, pencycuron, thiabendazole, thiophanate, thiophanate-methyl and zoxamide
(3) Inhibitors of the respiration, for example diflumetorim as CI-respiration inhibitor; bixafen, boscalid, carboxin, fenfuram, flutolanil, fluopyram, furametpyr, furmecyclox, isopyrazam (9R-component), isopyrazam (9S-component), mepronil, oxycarboxin, penthiopyrad, thifluzamide as CII-respiration inhibitor; amisulbrom, azoxystrobin, cyazofamid, dimoxystrobin, enestroburin, famoxadone, fenamidone, fluoxastrobin, kresoxim-methyl, metominostrobin, orysastrobin, picoxystrobin, pyraclostrobin, pyribencarb, trifloxystrobin as CIII-respiration inhibitor.
(4) Compounds capable to act as an uncoupler, like for example binapacryl, dinocap, fluazinam and meptyldinocap.
(5) Inhibitors of the ATP production, for example fentin acetate, fentin chloride, fentin hydroxide, and silthiofam.
(6) Inhibitors of the amino acid and/or protein biosynthesis, for example andoprim, blasticidin-S, cyprodinil, kasugamycin, kasugamycin hydrochloride hydrate, mepanipyrim and pyrimethanil.
(7) Inhibitors of the signal transduction, for example fenpiclonil, fludioxonil and quinoxyfen.
(8) Inhibitors of the lipid and membrane synthesis, for example biphenyl, chlozolinate, edifenphos, etridiazole, iodocarb, iprobenfos, iprodione, isoprothiolane, procymidone, propamocarb, propamocarb hydrochloride, pyrazophos, tolclofos-methyl and vinclozolin.
(9) Inhibitors of the ergosterol biosynthesis, for example aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulfate, imibenconazole, ipconazole, metconazole, myclobutanil, naftifine, nuarimol, oxpoconazole, paclobutrazol, pefurazoate, penconazole, piperalin, prochloraz, propiconazole, prothioconazole, pyributicarb, pyrifenox, quinconazole, simeconazole, spiroxamine, tebuconazole, terbinafine, tetraconazole, triadimefon, triadimenol, tridemorph, triflumizole, triforine, triticonazole, uniconazole, viniconazole and voriconazole.
(10) Inhibitors of the cell wall synthesis, for example benthiavalicarb, dimethomorph, flumorph, iprovalicarb, mandipropamid, polyoxins, polyoxorim, prothiocarb, validamycin A, and valiphenal.
(11) Inhibitors of the melanine biosynthesis, for example carpropamid, diclocymet, fenoxanil, phthalide, pyroquilon and tricyclazole.
(12) Compounds capable to induce a host defence, like for example acibenzolar-S-methyl, probenazole, and tiadinil.
(13) Compounds capable to have a multisite action, like for example bordeaux mixture, captafol, captan, chlorothalonil, copper naphthenate, copper oxide, copper oxychloride, copper preparations such as copper hydroxide, copper sulphate, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, oxine-copper, propamidine, propineb, sulphur and sulphur preparations including calcium polysulphide, thiram, tolylfluanid, zineb and ziram.
(14) Further compounds like for example 2,3-dibutyl-6-chlorothieno[2,3-d]pyrimidin-4(3H)-one, ethyl (2Z)-3-amino-2-cyano-3-phenylprop-2-enoate, N-[2-(1,3-dimethyl)butypphenyl]-5-fluoro-1,3-dimethyl-1H-pyrazole-4-carboxamide, N-{2-[1,1′-bi(cyclopropyl)-2-yl]phenyl}-3-(difluoromethyl)-1-methyl-1H-pyrazole-4-carboxamide, 3-(difluoromethyl)-1-methyl-N-(3′,4′,5′-trifluorobiphenyl-2-yl)-1H-pyrazole-4-carboxamide, 3-(difluoromethyl)-N-[4-fluoro-2-(1,1,2,3,3,3-hexafluoropropoxy)phenyl]-1-methyl-1H-pyrazole-4-carboxamide, (2E)-2-(2-{[6-(3-chloro-2-methylphenoxy)-5-fluoropyrimidin-4-yl]oxy}phenyl)-2-(methoxyimino)-N-methylethanamide, (2E)-2-{2-[({[(2E,3E)-4-(2,6-dichlorophenyl)but-3-en-2-ylidene]amino}oxy)methyl]phenyl }-2-(methoxyimino)-N-methylethanamide, 2-chloro-N-(1,1,3-trimethyl-2,3-dihydro-1H-inden-4-yl)pyridine-3-carboxamide, N-(3-ethyl-3,5,5-trimethylcyclohexyl)-3-(formylamino)-2-hydroxybenzamide, 5-methoxy-2-methyl-4-(2-{[({(1E)-1-[3-(trifluoromethyl)phenyl]-ethylidene}amino)oxy]methyl}phenyl)-2,4-dihydro-3H-1,2,4-triazol-3-one, (2E)-2-(methoxyimino)-N-methyl-2-(2-{[({(1E)-1-[3-(trifluoromethyl)phenyl]ethylidene}amino)-oxy]methyl}phenyl)ethanamide, (2E)-2-(methoxyimino)-N-methyl-2-{2-[(E)-({1-[3-(trifluoromethyl)phenyl]ethoxy}imino)methyl]phenyl}ethanamide, (2E)-2-{2-[({[(1E)-1-(3-{[(E)-1-fluoro-2-phenylethenyl]oxy}phenyl)ethylidene]amino}oxy)methyl]phenyl}-2-(methoxyimino)-N-methylethanamide, 1-(4-chlorophenyl)-2-(1H-1,2,4-triazol-1-yl)cycloheptanol, methyl 1-(2,2-dimethyl-2,3-dihydro-1H-inden-1-yl)-1H-imidazole-5-carboxylate , N-ethyl-N-methyl-N′-{2-methyl-5-(trifluoromethyl)-4-[3-(trimethylsilyl)propoxy]phenyl}imidoformamide, N′-{5-(difluoromethyl)-2-methyl-4-[3-(trimethylsilyl)propoxy]phenyl}-N-ethyl-N-methylimidoformamide, O-{1-[(4-methoxyphenoxy)methyl]-2,2-dimethylpropyl} 1H-imidazole-1-carbothioate, N-[2-(4-{[3-(4-chlorophenyl)prop-2-yn-1-yl]oxy}-3-methoxyphenyl)ethyl]-N²-(methylsulfonyl)valinamide, 5-chloro-7-(4-methylpiperidin-1-yl)-6-(2,4,6-trifluorophenyl)[1,2,4]triazolo[1,5-a]pyrimidine, 5-amino-1,3,4-thiadiazole-2-thiol, propamocarb-fosetyl, 1-[(4-methoxyphenoxy)methyl]-2,2-dimethylpropyl 1H-imidazole-1-carboxylate, 1-methyl-N-[2-(1,1,2,2-tetrafluoroethoxy)phenyl]-3-(trifluoromethyl)-1H-pyrazole-4-carboxamide, 2,3,5,6-tetrachloro-4-(methylsulfonyl)pyridine, 2-butoxy-6-iodo-3-propyl-4H-chromen-4-one, 2-phenylphenol and salts, 3-(difluoromethyl)-1-methyl-N-[2-(1,1,2,2-tetrafluoroethoxy)phenyl]-1H-pyrazole-4-carboxamide, 3,4,5-trichloropyridine-2,6-dicarbonitrile, 3-[5-(4-chlorophenyl)-2,3-dimethylisoxazolidin-3-yl]pyridine, 3-chloro-5-(4-chlorophenyl)-4-(2,6-difluorophenyl)-6-methylpyridazine, 4-(4-chlorophenyl)-5-(2,6-difluorophenyl)-3,6-dimethylpyridazine, quinolin-8-ol, quinolin-8-ol sulfate (2:1) (salt), 5-methyl-6-octyl-3,7-dihydro[1,2,4]triazolo [1,5-a]pyrimidin-7-amine, 5-ethyl-6-octyl-3 ,7-dihydro [1,2,4]triazolo [1,5-a]pyrimidin-7-amine, benthiazole, bethoxazin, capsimycin, carvone, chinomethionat, chloroneb, cufraneb, cyflufenamid, cymoxanil, cyprosulfamide, dazomet, debacarb, dichlorophen, diclomezine, dicloran, difenzoquat, difenzoquat methylsulphate, diphenylamine, ecomate, ferimzone, flumetover, fluopicolide, fluoroimide, flusulfamide, flutianil, fosetyl-aluminium, fosetyl-calcium, fosetyl-sodium, hexachlorobenzene, irumamycin, isotianil, methasulfocarb, methyl (2E)-2-{2-[({cyclopropyl[(4-methoxyphenyl)imino]methyl}thio)methyl]phenyl}-3-methoxyacrylate, methyl isothiocyanate, metrafenone, (5-bromo-2-methoxy-4-methylpyridin-3-yl)(2,3,4-trimethoxy-6-methylphenyl)methanone, mildiomycin, tolnifanide, N-(4-chlorobenzyl)-3-[3-methoxy-4-(prop-2-yn-1-yloxy)phenyl]propanamide, N-[(4-chlorophenyl)(cyano)methyl]-3-[3-methoxy-4-(prop-2-yn-1-yloxy)phenyl]propanamide, N-[(5-bromo-3-chloropyridin-2-yl)methyl]-2,4-dichloropyridine-3-carboxamide, N-[1-(5-bromo-3-chloropyridin-2-yl)ethyl]-2,4-dichloropyridine-3-carboxamide, N-[1-(5-bromo-3-chloropyridin-2-yl) ethyl]-2-fluoro-4-iodopyridine-3-carb oxamide, N-{(Z)-[(cyclopropyl-methoxy)imino][6-(difluoromethoxy)-2,3-difluorophenyl]methyl}-2-phenylacetamide, N-{(E)-[(cyclopropylmethoxy)imino][6-(difluoromethoxy)-2,3-difluorophenyl]methyl}-2-phenylacetamide, natamycin, nickel dimethyldithiocarbamate, nitrothal-isopropyl, octhilinone, oxamocarb, oxyfenthiin, pentachlorophenol and salts, phenazine-1-carboxylic acid, phenothrin, phosphorous acid and its salts, propamocarb fosetylate, propanosine-sodium, proquinazid, pyrrolnitrine, quintozene, S-prop-2-en-1-yl 5-amino-2-(1-methylethyl)-4-(2-methylphenyl)-3-oxo-2,3-dihydro-1H-pyrazole-1-carbothioate, tecloftalam, tecnazene, triazoxide, trichlamide, 5-chloro-N′-phenyl-N′-prop-2-yn-1-ylthiophene-2-sulfonohydrazide and zarilamid.

In a preferred embodiment the second fungicide is trifloxystrobin (methyl (E)-methoxyimino-{(E)-α-[1-(α,α,α-trifluoro-m-tolypethylideneaminooxy]-o-tolyl}acetate). In a more preferred embodiment of the invention a composition comprising of 3-difluoromethyl-1-methyl-1H-pyrazole-4-carboxylic acid [2-(2,4-dichlorophenyl)-2-methoxy-1-methyl-ethyl]-amide and trifloxystrobin is used for extending shelf life of fruits and vegetables, preferably of strawberries.

In a more preferred embodiment of the invention a composition comprising of 3-difluoromethyl-1-methyl-1H-pyrazole-4-carboxylic acid [2-(2,4-dichlorophenyl)-2-methoxy-1-methyl-ethyl]-amide and trifloxystrobin is used for extending shelf life of strawberries.

In a more preferred embodiment of the invention a composition comprising of 3-difluoromethyl-1-methyl-1H-pyrazole-4-carboxylic acid [2-(2,4-dichlorophenyl)-2-methoxy-1-methyl-ethyl]-amide and trifloxystrobin is used for extending shelf life of apricots.

In another preferred embodiment the second fungicide is azoxystrobin (methyl (2E)-2-(2-{[6-(2-cyanophenoxy)pyrimidin-4-yl]oxy}phenyl)-3-methoxyacrylate). In a more preferred embodiment of the invention a composition comprising of 3-difluoromethyl-1-methyl-1H-pyrazole-4-carboxylic acid [2-(2,4-dichlorophenyl)-2-methoxy-1-methyl-ethyl]-amide and azoxystrobin is used for extending shelf life of fruits and vegetables, preferably of strawberries.

In a more preferred embodiment of the invention a composition comprising of 3-difluoromethyl-1-methyl-1H-pyrazole-4-carboxylic acid [2-(2,4-dichlorophenyl)-2-methoxy-1-methyl-ethyl]-amide and azoxystrobin is used for extending shelf life of strawberries.

In a more preferred embodiment of the invention a composition comprising of 3-difluoromethyl-1-methyl-1H-pyrazole-4-carboxylic acid [2-(2,4-dichlorophenyl)-2-methoxy-1-methyl-ethyl]-amide and azoxystrobin is used for extending shelf life of apricots.

In another preferred embodiment the second fungicide is trifloxystrobin (methyl (E)-methoxyimino-{(E)-α-[1-(α,α,α-trifluoro-m-tolypethylideneaminooxy]-o-tolyl}acetate). In a more preferred embodiment of the invention a composition comprising of 3-difluoromethyl-1-methyl-1H-pyrazole-4-carboxylic acid [2-(2,4-dichlorophenyl)-2-methoxy-1-methyl-ethyl]-amide and trifloxystrobin is used for extending shelf life of fruits and vegetables, preferably of strawberries.

In a more preferred embodiment of the invention a composition comprising of 3-difluoromethyl-1-methyl-1H-pyrazole-4-carboxylic acid [2-(2,4-dichlorophenyl)-2-methoxy-1-methyl-ethyl]-amide and trifloxystrobin is used for extending shelf life of strawberries.

In a more preferred embodiment of the invention a composition comprising of 3-difluoromethyl-1-methyl-1H-pyrazole-4-carboxylic acid [2-(2,4-dichlorophenyl)-2-methoxy-1-methyl-ethyl]-amide and trifloxystrobin is used for extending shelf life of apricots.

A further embodiment of the present invention is a method extending shelf life of fruits and vegetables characterized in that, 3-difluoromethyl-1-methyl-1 H-pyrazole-4-carboxylic acid [2-(2,4-dichlorophenyl)-2-methoxy-1-methyl-ethyl]-amide was applied to the crop prior to the harvest of the fruits and vegetables.

### EXAMPLES

A Control in apricots

Methods

An apricot fungicide trial is conducted by at an apricot grower orchard. Apricot (Prunus spp.) plots are established with tree plots arranged in a randomized complete block design with replications each. For the treatments standard grower spray equipment and spray volumes are used as well as standard products for comparison.

Fruit are harvested and packed into fruit boxes. Fruit boxes are transported in bags and are placed in the laboratory in cool storage in bags. Boxes are removed from bags and examined for incidence of disease at different time points after harvest. Data is presented as percent incidence for the post harvest diseases that naturally occurred.

B Control in Strawberries

Methods

A strawberry fungicide trial is conducted as Strawberry (Fragaria spp.) plots are with several rows per plot arranged in a randomized complete block design with replications each. Treatments are prepared in a standard water volume and applied to plots with a backpack sprayer.

All ripe berries are collected from the plots several times first to establish baseline disease rating and thereafter several times per week (approximately 1 to 5 days after each application) and are incubated on paper towels in separate plastic storage boxes at app. 17°Celsius.

Boxes are rated for the three post harvest diseases that is developed by counting the number of infected berries per box. As berries melt from disease they are removed from each box to reduce within box contamination between berries. Data are recorded and graphed as cumulative incidence of disease like Rhizopus stolonifer Botrytis cinerea, Penicillium spp., and cumulative total disease.

1. N phenylethylpyrazole carboxamide derivative and/or Salt thereof capable of extending shelf life of a fruit and/or vegetable, of formula (I) 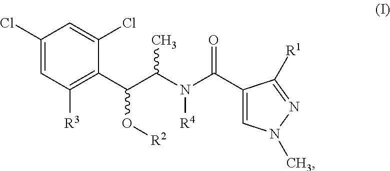
wherein
R¹ represents a halo(Ci-C₆)alkyl group,
R² represents a (Ci-C₆)alkyl group,
R³ represents a hydrogen or halogen atom,
R⁴ represents hydrogen or a (C₃-C₆)cycloalkyl group.

2. N-phenylethylpyrazole carboxamide derivative and/or salt according to claim 1, wherein in formula (I)
R¹ represents CHF₂ or CF₃;
R² represents methyl or ethyl;
R³ represents hydrogen or chloro;
R⁴ represents hydrogen or cyclopropyl.

3. N-phenylethylpyrazole carboxamide derivative and/or salt according to claim 1, which is 3-difluoromethyl-1-methyl-1H-pyrazole-4-carboxylic acid [2-(2,4-dichlorophenyl)-2-methoxy-1-methyl-ethyl]-amide or a derivatives and/or salt thereof.

4. N-phenylethylpyrazole carboxamide derivative and/or salt according to claim 1, wherein the fruit is selected from the group consisting of banana, blackcurrant, redcurrant, gooseberry, tomato, eggplant, guava, lucuma, chili pepper, pomegranate, kiwifruit, grape, table grape, pumpkin, gourd, cucumber, melon, orange, lemon, lime, grapefruit, banana, cranberry, blueberry, blackberry, raspberry, boysenberry, hedge apple, pineapple, fig, mulberry, apple, apricot, peach, cherry, green bean, sunflower seed, strawberry and plum.

5. N phenylethylpyrazole carboxamide derivative and/or salt according to claim 1, wherein the vegetable is selected from the group consisting of broccoli, cauliflower, globe artichokes, sweetcorn, maize, kale, collard greens, spinach, beet greens, turnip greens, endive; leeks, Brussels sprouts, celery, rhubarb, asparagus, ginger; potatoes, Jerusalem artichokes, sweet potato, yam bean sprouts, carrots, parsnips, beets, radishes, turnips, onions, garlic, and shallots.

6. N phenylethylpyrazole carboxamide derivative and/or salt according to claim 1, wherein the fruit comprises strawberries.

7. N phenylethylpyrazole carboxamide derivative and/or salt according to claim 1, wherein the N-phenylethylpyrazole carboxamide derivative and/or salt thereof has been applied to a crop prior to harvest of the fruit or vegetable.

8. N-phenylethylpyrazole carboxamide derivative and/or salt according to claim 1, said N-phenylethylpyrazole carboxamide derivative and/or salt thereof is applied to a crop at a rate ranging from 1 to 250 g/ha—based upon pure a.s.

9. A composition comprising said N-phenylethylpyrazole carboxamide derivative and/or salt according to claim 1, and a further fungicide, and wherein said composition has been applied to a crop and/or to a fruit and/or vegetable thereof.

10. A composition according to claim 9, wherein the composition comprises 3-difluoromethy 1-1-methyl-1H-pyrazole-4-carboxylic acid [2-(2,4-dichlorophenyl)-2-methoxy-1-methyl-ethyl]-amide or a derivative and/or salt thereof and trifloxystrobin (methyl (E,)-methoxyimino-{(E,)-a-[1-(a,a,a-trifluoro-m-tolyl)ethylideneaminooxy]-o-tolyl}acetate).

11. A composition to claim 9, wherein the composition comprises 3-difluoromethy 1-1-methyl-1H-pyrazole-4-carboxylic acid [2-(2,4-dichlorophenyl)-2-methoxy-1-methyl-ethyl]-amide or a derivative and/or salt thereof and azoxystrobin (methyl (2E)-2-(2-{[6-(2-cyanophenoxy)pyrimidin-4-yl]oxy}phenyl)-3-methoxyacrylate).

12. A method for extending shelf life of a fruit and/or vegetable, comprising applying a N-phenylethylpyrazole carboxamide derivative and/or salt thereof to a crops prior to harvest of the fruit or vegetable.

13. A method according to claim 12, wherein, the N-phenylethylpyrazole carboxamide derivative is 3-difluoromethyl-1-methyl-1H-pyrazole-4-carboxylic acid [2-(2,4-dichlorophenyl)-2-methoxy-1-methyl-ethyl]-amide or a derivative and/or salt thereof.
